(19) United States
(12) Patent Application Publication
(10) Pub. No.: US 2008/0102116 A1
(43) Pub. Date: May 1, 2008

(54) Quick Dissolve Medicament and Method of Manufacturing

(75) Inventors: Ronald L. Perry, Paw Paw, MI (US); Rick Jacobs, Allegan, MI (US); Harold W. Reick, Grand Rapids, MI (US)

Correspondence Address: PRICE HENEVELD COOPER DEWITT & LITTON, LLP, 695 KENMOOR, S.E., P O BOX 2567, GRAND RAPIDS, MI, 49501, US

(73) Assignee: Perrigo Company

(21) Appl. No.: 11/662,773

(22) PCT Filed: Sep. 8, 2005
(86) PCT No.: PCT/US05/31962

§ 371 Date: Mar. 12, 2007

Related U.S. Application Data
(60) Provisional application No. 60/609,313, filed on Sep. 13, 2004.

(51) Int. Cl. A61K 9/20; A61K 9/46; A61K 31/167

(52) U.S. Cl. 424/465; 424/466; 424/464; 514/629

(57) ABSTRACT

A medicament core which contains a super disintegrant or an effervescent couple including a foaming agent and a pharmaceutically acceptable acid activator, which core is partially covered by preformed shell coverings shaped to expose at least a part of the core for activation upon exposure to bodily fluids.

### CROSS-REFERENCE TO RELATED APPLICATIONS

This application claims priority under 35 U.S.C. § 119(e) on U.S. Provisional Application No. 60/609,313 entitled QUICK DISSOLVE CAPSULE AND METHOD OF MANUFACTURING, filed on Sep. 13, 2004, by Ronald L. Perry et al., the entire disclosure of which is incorporated herein by reference.

### BACKGROUND OF THE INVENTION

The present invention relates to a press-fit fast release caplet having gelatin covered ends leaving an uncovered center band.

Gelatin covered caplets have become a popular dosage form for medicaments and provide tamper-resistant safety as well as easy swallowability. Several methods have evolved for the gelatin covering of caplets, including the dip-coating of caplets with a gelatin solution, the encapsulating of gelatin capsules utilizing an encapsulation machine and process as disclosed in U.S. Pat. No. 6,209,296 and in copending U.S. patent application Ser. No. 10/899,924, filed on Jul. 27, 2004, entitled TABLET ENCAPSULATING MACHINE. Also, caplets have been employed in which gelatin shells are hydrated and subsequently shrink-fitted onto a caplet to provide a caplet core which is fully enclosed by gelatin capsule shells. U.S. Pat. Nos. 5,415,868 and 5,824,338 are examples of such dosage forms.

In order for a medicament to enter the bloodstream of a patient, it is necessary for the gelatin covering of gelatin covered medicaments to dissolve, typically in the stomach, which takes a certain period of time before the medicament can effectively be assimilated by the person's body. Although uncoated medicaments are faster acting, they tend to be less easily swallowed than medicaments having a gelatin covering.

It would be desirable, therefore, to provide a medicament which has the ease of swallowability of a gelatin-covered caplet and yet the relatively rapid release of medicament as in uncoated caplets.

### SUMMARY OF THE INVENTION

The medicament of the present invention and its method of manufacture solves this need by providing a medicament core which contains a super disintegrant or an effervescent couple including a foaming agent and a pharmaceutically acceptable acid activator, which core is partially covered by a gelatin covering such that at least a part of the core is exposed for activation upon exposure to bodily fluids.

In the preferred embodiment of the invention, the medicament core is a caplet shaped core with gelatin capsule shells press-fit from opposite ends thereof, leaving a gap exposing the core between the facing ends of the gelatin capsule shells. Also in the preferred embodiment of the invention, the gelatin capsule shells are press-fit onto opposite ends of the caplet core, leaving a gap of from about 3 to about 4 mm between the facing ends of the gelatin capsule shells. The medicament, therefore, provides a gelatin covering for at least part of the medicament and an exposed core which causes the caplet to split and rapidly dissolve to release its active ingredients before the gelatin dissolves, resulting in a quick accessibility of the medicament to the patient.

These and other features, objects and advantages of the present invention will become apparent upon reading the following description thereof together with reference to the accompanying drawings.

### BRIEF DESCRIPTION OF THE DRAWINGS

FIG. 1 is an exploded perspective view of a medicament embodying the present invention;

FIG. 2 is a top plan view of the assembled medicament shown in FIG. 1;

FIG. 3 is an end view of one of the gelatin capsule shells;

FIG. 4 is a block diagram of the method of manufacturing the medicament shown in FIGS. 1 and 2;

FIG. 5 is an exploded perspective view of an alternative form of a medicament embodying the present invention; and

FIG. 6 is a side elevational view of the medicament of FIG. 5.

### DETAILED DESCRIPTION OF THE PREFERRED EMBODIMENT

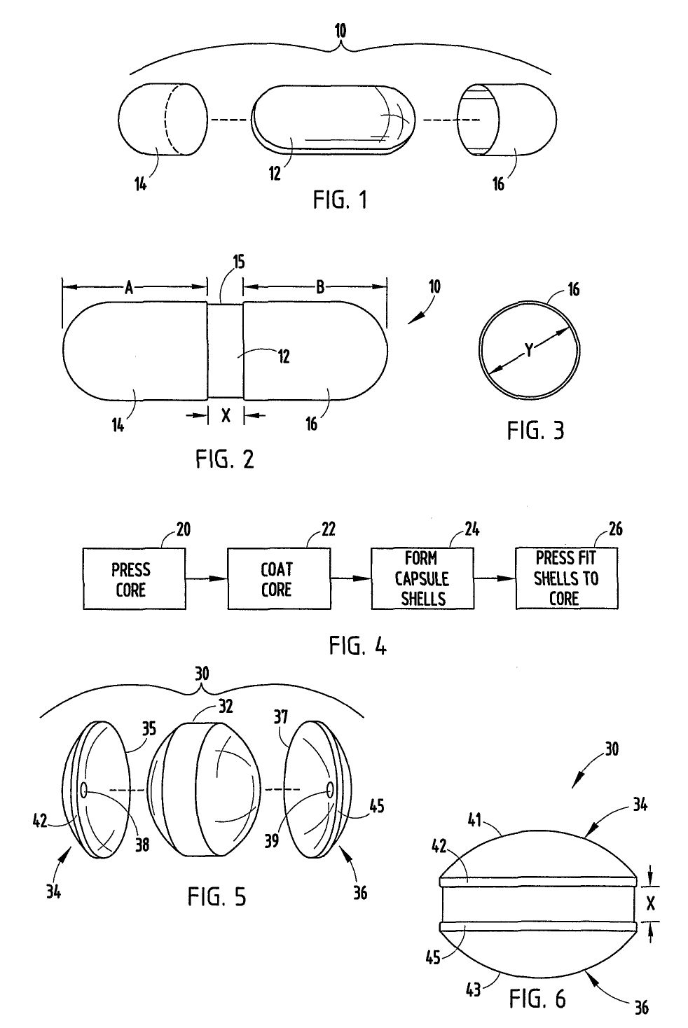

Referring initially to FIG. 1, there is shown a medicament 10 embodying the present invention which is in the form of caplet-shaped core 12, a first gelatin capsule shell 14, and a second gelatin capsule shell 16 for partially encapsulating the core 12. As seen in FIG. 2, the capsule shells 14 and 16 do not abut when press-fitted onto core 12 but rather leave a gap having a width identified by the dimension X in FIG. 2 of from about 3 to 4 mm exposing the core to bodily fluids when the medicament is taken by a patient. The capsule shells 14 and 16 are conventional gelatin capsules which are commercially available but which are shorter than a typical capsule shell to leave the exposed peripheral band 15 of core 12, as seen in FIG. 2.

In one embodiment of the invention, the core 12 had an overall length of about 0.85 inches, with each capsule shell 14 and 16 having a length indicated by dimensions A and B in FIG. 2 of about 0.356 inches. Band 15, dimension “X” in this embodiment, was about 0.138 inches. Although capsule shells 14, 16 are preferably of equal length, they could be of different lengths, thus shifting the peripheral band 15 along the longitudinal axis of the core 12. Core 12 can contain any desired active ingredient as the medicament. As an example only, the core 12 may contain an analgesic including non-steroidal anti-inflammatory drugs (NSAIDs) including but not limited to aspirin, ibuprofen, acetaminophen, naproxen sodium, and the like, or combinations of such medicaments with antihistamines such as chlorpheniramine maleate, dextromethorphan, pseudoephedrine, or anti-tussive agents. The medicament of core 12, however, is not limited to these specific examples. In addition to the active ingredients and typical excipients and binders for compactability, core 12 also includes one of a super disintegrant or an effervescent couple typically comprising a foaming agent, such as bicarbonate of soda, and a pharmaceutically acceptable acid activator, such as citric acid. Examples of core materials are given below:

### EXAMPLE 1

| Ingredients | mg/tab | % |
|---|---|---|
| APAP Compap Course L | 555.500 | 88.174603 |
| (90% acetaminophen) | | |
| Copovidone S-630 | 11.500 | 1.825397 |
| Crospovidone XL | 63.000 | 10.000000 |

### EXAMPLE 2

| Ingredients | mg/tab | % |
|---|---|---|
| APAP Compap Course L | 555.500 | 87.757 |
| (90% acetaminophen) | | |
| Microcryst Cellulose | 14.500 | 2.291 |
| Crospovidone XL | 63.000 | 9.953 |

### EXAMPLE 3

| Ingredients | mg/tab | % |
|---|---|---|
| APAP Compap Course L | 555.500 | 81.811 |
| (90% acetaminophen) | | |
| Microcryst Cellulose | 14.500 | 2.135 |
| Sod Bicarb #2 F-gran | 54.500 | 8.027 |
| Citric Acid Anhydrs | 54.500 | 8.027 |

### EXAMPLE 4

| Ingredients | mg/tab | % |
|---|---|---|
| APAP Compap Course L | 555.500 | 77.910 |
| (90% acetaminophen) | | |
| Microcryst Cellulose | 14.500 | 2.034 |
| Crospovidone XL | 34.000 | 4.769 |
| Sod Bicarb #2 F-gran | 54.500 | 7.644 |
| Citric Acid Anhydrs | 54.500 | 7.644 |

The core, as shown in FIG. 4, is manufactured in step 20 by a typical tableting press, which compresses the active and inactive ingredients into preferably an elongated caplet-shaped core 12, as seen in FIG. 1, although other tablet forms may also be employed. Upon the forming of the core, typically it is coated with a hydroxy propylmethocellulose (HPMC) or hydroxy propocellulose (HPC) to provide a protective coating and add surface strength to the core for subsequent handling. The coating step 22 is typically accomplished by spray coating, pan coating, or the like. Subsequently, the capsule shell halves 14 and 16 are formed, as shown by step 24 in FIG. 4, typically by dip-coating pins in baths of gelatin to a depth (in one embodiment) of approximately 0.356 inches with an internal diameter as shown by dimension Y in FIG. 3, for a core of approximately 0.25 inches in diameter of about 0.256 inches +/−0.003 inches. The capsule shells 14, 16 will typically be of different colors which function to identify the type of medicament involved.

The thickness of the capsule shells 14 and 16 is conventional. Shells 14 and 16 are subsequently press-fit onto the core 12 as illustrated by step 26 in FIG. 4, utilizing a commercially available press-fit machine, such as one available from I.M.A. North America Model No. Zanasi 70c. During the press-fitting process, the shortened capsule shells 14 and 16 leave the peripheral band 15, as seen in FIG. 2, exposing the core 12 to the bodily fluids upon swallowing medicament 10. This band 15 of exposed core rapidly dissolves under the influence of the super disintegrant or effervescent couple to break the medicament 10 into halves at the center location of band 15 prior to dissolving of gelatin core capsule shells 14 and 16, thereby allowing the active ingredient contained within core 12 to be rapidly assimilated by the body for providing quick relief from symptoms for which the medicament is being taken.

The exact dimensions of the caplet and gelatin capsule shells can be varied as long as they interfit with one another to press-fit or otherwise attach the capsule shell halves to the core in such a manner as the core includes an exposed area. Further, the medicament can be manufactured with an elongated capsule shell which leaves one end of the medicament exposed as opposed to a center band, although the center band is preferred. Further, other shapes of tablet cores may be employed with a suitable gelatin covering which exposes a sufficient surface area of the medicament such that the super disintegrant or effervescent couple will effectively release the active ingredients into the body more quickly than an entirely gelatin covered medicament. Also, capsule shell halves 14 and 16 can be made of materials other than gelatin. Such materials include inter alia polyvinyl alcohol, starches, alginates, acrylates, polyvinyl pyrrolidone, cellulose derivates, and polysiloxanes.

Referring now to FIGS. 5 and 6, there is shown an alternative dosage form 30 which incorporates a conventional tablet-shaped core 32 which is conventionally press manufactured by compacting the pharmaceutical active ingredients and excipients, such as employed in the caplet-shaped core 12. The core is partially covered by a pair of shells 34 and 36, which are formed by a molding or stamping process generally in the shape of hemispheres which have truncated peripheral edges 35 and 37, respectively, which leaves a gap identified by reference X in FIG. 6 of from 3 mm to 4 mm for exposing the edge of core 32 to bodily fluids when administered. In order to adhere the shells 34, 36 to core 32, a pharmaceutically acceptable adhesive 38, 39 is applied to the interior surface of the shells 34, 36 prior to the shells being placed over core 32 for adhering the shells to the core. The adhesive 38, 39 can be dots of liquid gelatin, which are appropriately placed within the shells or other pharmaceutically acceptable adhesive and may be applied by a dropper or other conventional methods for applying a liquid adhesive. Alternatively, an adhesive coating can be spray coated on the interior of the preformed gelatin shells 34, 36, after which they are pressed onto the shell 32 on opposite sides thereof and allowed to dry. The shells 34, 36 may have a moisture content after forming which allows them to readily fit over core 32 and, upon curing the adhesive and drying the shells, they tend to shrink onto and assist the adhesive in holding the shells in tight engagement with the core 32. Depending on the core shape, the generally hemispherical shells 34, 36 may include a dome section, such as 41 and 43, respectively, and a vertically extending band section 42 and 45, respectively, as shown in FIG. 6. The material of shells 34, 36 can be the same as that disclosed in the embodiment of FIGS. 1-4, as are the ingredients of the core 32. As in the first embodiment shells 34, 36 may be of different colors to color code the type of medicament being partially covered by the shells.

It will become apparent to those skilled in the art that various modifications to the preferred embodiments of the invention as described herein can be made without departing from the spirit or scope of the invention as defined by the appended claims.

The invention claimed is:

1. A quick dissolve medicament comprising:
a medicament core including an active ingredient and excipients for rapidly releasing the active ingredient, said core at least partially covered by a press-fit covering which leaves an area of the core exposed.

2. The medicament as defined in claim 1 wherein said core is caplet shaped.

3. The medicament as defined in claim 2 wherein said covering comprises a pair of capsule shells.

4. The medicament as defined in claim 3 wherein said capsule shells have a combined length less than the length of said caplet shaped core such that a peripheral band of said core is exposed.

5. The medicament as defined in claim 4 wherein said peripheral band is from about 3 mm to about 4 mm in length.

6. The medicament as defined in claim 4 wherein said capsule shells are made of gelatin.

7. The medicament as defined in claim 1 wherein said core is tablet shaped.

8. The medicament as defined in claim 7 wherein said covering comprises a pair of generally hemispherical shells.

9. The medicament as defined in claim 8 wherein said shells are adhered to said core by a pharmaceutically acceptable adhesive.

10. The medicament as defined in claim 9 wherein said adhesive is gelatin based.

11. A method of manufacturing a medicament comprising:
pressing a medicament core including an active ingredient and one of a super disintegrant and a effervescent couple in a caplet shape;
coating the core with a pharmaceutically acceptable coating;
forming capsule shell halves with a length less than one-half the length of the caplet core; and
press-fitting the capsule shells onto opposite ends of the core, leaving an exposed peripheral band.

12. The method as defined in claim 11 wherein said pressing step comprises using a super disintegrant crosspovidone.

13. The method as defined in claim 11 wherein said pressing step comprises using an effervescent couple comprising a foaming agent and an activator.

14. The method as defined in claim 12 wherein said foaming agent is sodium bicarbonate and said activator is citric acid.

15. The method as defined in claim 11 wherein said coating step comprises coating said core with one of HPMC and HPC.

16. The method as defined in claim 11 wherein said forming step comprises forming capsule shells from gelatin.

17. The method as defined in claim 16 wherein said press-fitting step leaves an exposed peripheral band of core of from about 3 mm to about 4 mm in length.

18. A medicament comprising:
an elongated caplet having a core including an active ingredient and one of a super disintegrant and a effervescent couple; and
a pair of capsule shells press-fitted on opposite ends of said caplet, said shells having a length selected to leave an exposed peripheral band of the core between facing ends of said capsule shells.

19. The medicament as defined in claim 18 wherein said peripheral band is from about 3 mm to about 4 mm in length.

20. The medicament as defined in claim 19 wherein said capsule shells are made of gelatin.

21. The medicament as defined in claim 18 wherein said super disintegrant is crosspovidone.

22. The medicament as defined in claim 21 wherein said crosspovidone is crosspovidone XL.

23. The medicament as defined in claim 18 wherein said effervescent couple is a foaming agent and an acid activator.

24. The medicament as defined in claim 23 wherein said foaming agent is sodium bicarbonate and said acid activator is citric acid.

25. A rapidly disintegratable orally administrable tablet comprising:
a compressed core including an effective amount of a pharmaceutically active ingredient, a binder and a disintegrant; and
a pair of gelatin shells attached to opposite sides of said core, said shells having a dimension such that they do not fully cover the core to leave an area of said core which is exposed.

26. The rapidly disintegratable tablet of claim 25 wherein the binder comprises at least one of microcrystalline cellulose and copovidone.

27. The rapidly disintegratable tablet of claim 25 wherein the disintegrant is crosspovidone.

28. The rapidly disintegratable tablet of claim 25 wherein the pharmaceutically active ingredient is acetaminophen.

29. The rapidly disintegratable tablet of claim 25 wherein the binder is microcrystalline cellulose, the disintegrant is crosspovidone, and the pharmaceutically active ingredient is acetaminophen.

30. The rapidly disintegratable tablet of claim 25 further comprising an effervescent system.

31. The rapidly disintegratable tablet of claim 30 wherein the effervescent system comprises a carbonate source and a pharmaceutically acceptable acid.

32. The rapidly disintegratable tablet of claim 31 wherein the carbonate source is sodium bicarbonate, and the pharmaceutically acceptable acid is citric acid.

33. The rapidly disintegratable tablet of claim 25 wherein the binder is microcrystalline cellulose, the disintegrant is crosspovidone, the pharmaceutically active ingredient is acetaminophen, and the tablet further comprises an effervescent system including a carbonate source and a pharmaceutically acceptable acid.

34. A rapidly disintegratable orally administrable tablet comprising:
a core consisting essentially of an effective amount of a pharmaceutically active ingredient, a binder, and a disintegrant; and
a pair of shells shaped for covering part of said core to improve swallowability and mouth feel, but which allow immediate contact of the core with fluid in the gastrointestinal tract upon oral ingestion.

35. The rapidly disintegratable tablet of claim 34 wherein said core is caplet-shaped.

36. The rapidly disintegratable tablet of claim 35 wherein said shells are press-fit over said caplet-shaped core.

37. The rapidly disintegratable tablet of claim 34 wherein said core is tablet-shaped.

38. The rapidly disintegratable tablet of claim 37 wherein said shells are generally hemispherical and truncated at a peripheral edge.

39. The rapidly disintegratable tablet of claim 38 wherein said shells are adhered to said tablet-shaped core by a pharmaceutically acceptable adhesive.

40. The rapidly disintegratable tablet of claim 39 wherein said adhesive is gelatin based.
